(19) United States
(12) Patent Application Publication
(10) Pub. No.: US 2010/0147309 A1
(43) Pub. Date: Jun. 17, 2010

(54) Tracheal Catheter With a Flexible Lumen for Subglottic Suctioning

(76) Inventors: Brian J. Cuevas, Cumming, GA (US); Michael Sleva, Atlanta, GA (US); Joseph A. Cesa, Cumming, GA (US); Scott M. Teixeira, Cumming, GA (US); David Robson, Riverside, RI (US)

Correspondence Address: KIMBERLY-CLARK WORLDWIDE, INC.;Tara Pohlkotte, 2300 Winchester Rd., NEENAH, WI, 54956, US

(21) Appl. No.: 12/334,016

(22) Filed: Dec. 12, 2008

(51) Int. Cl. A61M 16/04

(52) U.S. Cl. 128/207.14

(57) ABSTRACT

There is provided a tracheal catheter designed so that a suction catheter may be used to suction out any secretions that accumulate in the zone above the balloon. There is further provided a tracheal catheter having a ventilation lumen with a flexible wall to provide a flexible lumen which takes up negligible volume when the catheter is not inserted. The suction catheter may be inserted into the flexible lumen, bending the flexible lumen wall that normally lies substantially flat against the ventilating lumen. As the suction catheter is inserted into the lumen, the lumen opens to accept it. When the catheter is removed, the wall of the flexible lumen moves back into position against the ventilating lumen wall, again taking up negligible volume. There is also provided a method of suctioning the subglottic space of an intubated patient. This involves providing a tube as described above, inserting a catheter into the flexible lumen from the proximal end, and providing suction to the proximal end of the catheter.

### BACKGROUND

Tracheal catheters are used to assist patient breathing during and after medical procedures until they are able to breathe successfully on their own and be removed from assisted breathing. The catheters are connected to ventilators or respirators for mechanical ventilation of the lungs. The ventilator unit is connected to a hose set; the ventilation tubing or tubing circuit, delivering the ventilation gas to the patient.

There are two principle types of tracheal catheters; the endotracheal tube and the tracheostomy tube (trach tube). The endotracheal tube (ET tube) is inserted through the mouth of a patient and guided past the vocal cords and glottis into the trachea. A trach tube is inserted directly into the trachea through a stoma created in the throat and tracheal wall by surgical means and enters the trachea below the glottis. Both types of tube have a relatively large main ventilating lumen that delivers the air from the mechanical ventilating device to the lungs. A medical practicioner will normally choose the tube with the largest internal diameter possible relative to the size of the patient's trachea and airway. This minimizes the pressure drop through the tube and allows for the best mechanical ventilation possible.

As a result of this perceived problem with ET tubes, tracheostomy operations are becoming increasingly common and are being performed earlier in the patient's hospital stay in order to reduce the occurrence of VAP. In a tracheostomy operation, a trach tube is inserted directly into the trachea through a stoma created in the throat and tracheal wall by surgical means. The trach tube, therefore, enters the trachea below the glottis and trach tubes are believed to result in lower secretion rates and hence lower VAP rates. As a result of this placement, trach tube patients may be awake and alert after the insertion of the tube and may lead relatively normal lives, all the while having a trach tube in place.

The two types of tracheal catheters have a balloon (also called a cuff) located at or near their lower or distal end, surrounding the main ventilating lumen to ensure positive pressure ventilating of the lungs. The purpose of the balloon is to block the balance or remainder of the trachea so that ventilating air being delivered to the patient by the catheter does not merely leave the distal end of the catheter, reverse course and travel up the trachea to escape from the patient. The balloon ensures that the air must travel into the patient's lungs. The mechanical ventilator then reverses the flow of the air and it is removed from the patient via the same catheter, thus permitting positive pressure ventilation of the lungs. The balloon also aids in supporting the tube inside the trachea since its periphery lies against the internal lining of the trachea.

ET tubes are generally used for a number of days before a decision is made to switch a patient to a tracheostomy tube. Endotracheal tubes have been linked in some studies to an increased rate of ventilator acquired pneumonia (VAP). The reason for this is believed to be secretions from the oral and nasal cavities and in some cases stomach reflux, that then flow downward and pool above the cuff or leak past the cuff to the lungs.

A number of proposals have been made to improve the balloons so that secretions do not readily pass by them and travel to the lungs. U.S. Pat. Nos. 6,526,977 and 6,802,317 to Gobel, for example, teach oversized balloons with a wall thickness so low that the balloon walls lie in folds against the tracheal wall and the folds are so small that secretions cannot pass through them and travel on to the lungs. This solution inhibits and/or arrests the passage of secretions beyond the cuff, but does not provide for removal of secretions, for example, prior to extubation.

Removing the secretions within the trachea above the balloon has been another approach taken to reduce the likelihood of secretions entering the lungs. An endotracheal tube having a single dedicated suction lumen is available from Mallinckrodt Inc. This tube allows for the connection of a suction source to the suction lumen for evacuation of secretions above the cuff. While this approach works adequately there remains the possibility that the suction lumen can become clogged with secretions and be rendered unusable, particularly if suctioning is done on an intermittent basis.

A further and very important problem with known means of suctioning in the subglottic space is that the cross-sectional area available for the insertion of various suction catheters and other devices is very limited. The trachea is relatively small and a tube must be even smaller so that it may bend during insertion and during use. The path along which the tube is inserted is tortuous and bends back and forth is a slight “S” shape as it passes the vocal cords, making insertion challenging. If an additional fixed or dedicated lumen is added to a tube for the purpose of suctioning or rinsing, the outer diameter of the tube must be increased, increasing the risk of tracheal trauma during insertion, or the inner diameter, e.g. the ventilating lumen, must be made smaller. A dedicated lumen can also cause a decrease in the flexibility of the endotracheal tube, making insertion more difficult. The largest ventilating lumen possible is desired since this produces the least resistance to air flow (or pressure drop) through the tube. It is therefore with great reluctance that part of the cross-sectional area of the tube that may be used for ventilating the lungs is given over to other purposes.

In commercially available conventional endotracheal tubes having relatively thick cuffs, secretions pass the cuff into the lungs of a patient relatively easily. If these tubes are provided with suctioning lumens, and not all are, there must be continuous suction applied or some of the secretions will reach the lungs. Continuous suctioning, as discussed above, requires a dedicated suction lumen that takes valuable space from the ventilating lumen or requires a larger tube outer diameter. Continuous suctioning also has the potential to damage the trachea since it is possible for the suction lumen inlet to adhere itself to the tracheal wall, subjecting the sensitive tracheal wall tissue to the force of the suction used.

It would therefore be desirable to provide a tracheal tube and balloon design that allows secretions to be suctioned from above the cuff on an intermittent basis in order to reduce the possibility of the patient developing ventilator acquired pneumonia and damaging the trachea by suctioning the tube to the tracheal wall. It is also desirable to provide a means of removing secretions from the subglottic space where that means takes up a minimum of the area of the ventilating lumen. It is also desirable to provide these functions in a way that minimizes the likelihood of clogging of the suction lumen.

### SUMMARY

There is provided a tracheal catheter designed so that a suction catheter may be used to suction out any secretions that accumulate in the zone above the balloon (the subglottic space). There is further provided a tracheal catheter having a ventilation lumen with a flexible divider or wall adjacent to the ventilation lumen wall. The area between the ventilation lumen wall and the flexible lumen wall define the flexible lumen into which the suction catheter may be inserted. The flexible lumen takes up negligible volume when the suction catheter is not present, allowing the tracheal catheter to be comparable in internal diameter to a similar tracheal catheter without a flexible suction lumen.

In practice, the suction catheter may be inserted into the flexible lumen, bending the flexible lumen wall that normally lies substantially flat against the ventilating lumen. As the suction catheter is inserted into the flexible lumen, the flexible lumen wall bends away from the ventilating lumen wall to accept it. When the catheter is removed, the wall of the flexible lumen moves back into position against the ventilating lumen wall, again taking up negligible volume. The cross-sectional area of the ventilating lumen decreases momentarily while the suction catheter is inserted.

There is also provided a method of suctioning the subglottic space of an intubated patient. This involves providing a tracheal catheter as described above, inserting a suction catheter into the flexible lumen from the proximal end of the tracheal catheter until the distal end of the suction catheter reaches the port adjacent to and above the cuff near the distal end of the tracheal catheter, and providing suction to the proximal end of the suction catheter.

### BRIEF DESCRIPTION OF THE DRAWINGS

FIG. 1 is an illustration of a cuffed endotracheal tube.

FIG. 2 is an illustration of a cuffed tracheostomy tube.

FIG. 3 is a cross-sectional view of taken along line A-A of FIG. 1, showing the ventilation lumen, inflation lumen and flexible walled lumen where the flexible walled lumen is on the inside of the ventilation lumen.

FIG. 4 is a cross-sectional view of taken along line B-B of FIG. 2, showing the ventilation lumen, inflation lumen and flexible walled lumen where the flexible walled lumen is on the outside of the ventilation lumen.

FIG. 5 is a cross-sectional view of taken along line A-A of FIG. 1, showing the ventilation lumen, inflation lumen and flexible walled lumen where the flexible walled lumen is on the inside of the ventilation lumen and a suction catheter has been inserted into the flexible walled lumen.

FIG. 6 is a cross-sectional view of taken along line B-B of FIG. 2, showing the ventilation lumen, inflation lumen and flexible walled lumen where the flexible walled lumen is on the outside of the ventilation lumen and a suction catheter has been inserted into the flexible walled lumen.

### DETAILED DESCRIPTION

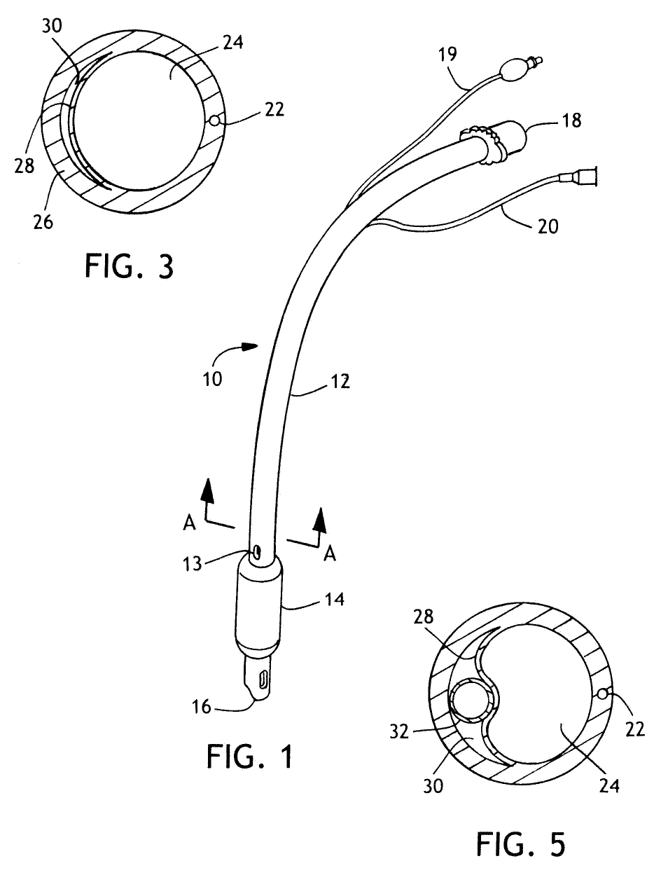

The tracheal catheter described herein can be better understood with reference to FIGS. 1-6, some of which illustrate exemplary embodiments as described below.

FIG. 1 is an illustration of a cuffed endotracheal device 10 having a tube 12 and an inflatable cuff 14. The endotracheal device 10 has a distal end 16 and a proximal end 18. The cuff 14 is inflated by air supplied to an inflation line 20. The endotracheal device 10 may connect to a ventilator (not shown) at its proximal end 18 so that breathing air may be delivered to a patient. The tube 12 has a flexible lumen 30 (not visible in FIG. 1) that terminates at a port 13 above the cuff 14 and is accessible through a flexible lumen access tube 19 that begins near the proximal end 18 of the tube 12. FIG. 3 is a cross sectional view of the tube 12 taken at point A-A showing the various lumens within the tube 12. This view illustrates the inflation lumen 22 that is connected on one end to the inflation line 20 and on the other end to the cuff 14 and that delivers the air from the inflation line 20 to the cuff 14. FIG. 3 also shows the ventilating lumen 24 that connects to the ventilator at the proximal end 18 of the tube. The ventilating lumen 24 runs the entire length of the tube 12 in order to deliver breathing air to the patient. The ventilating lumen 24 is enclosed by the ventilating lumen wall 26 and in part by the flexible wall 28 which is adjacent the ventilating lumen wall 26. A flexible, collapsible lumen 30 is formed between the ventilating lumen wall 26 and the flexible wall 28 on the opposite side of the flexible lumen wall 28 from the ventilating lumen 24. The flexible lumen 30 terminates and opens onto the outside of the tube 12 just above the cuff 14 where it forms an aperture or port 13.

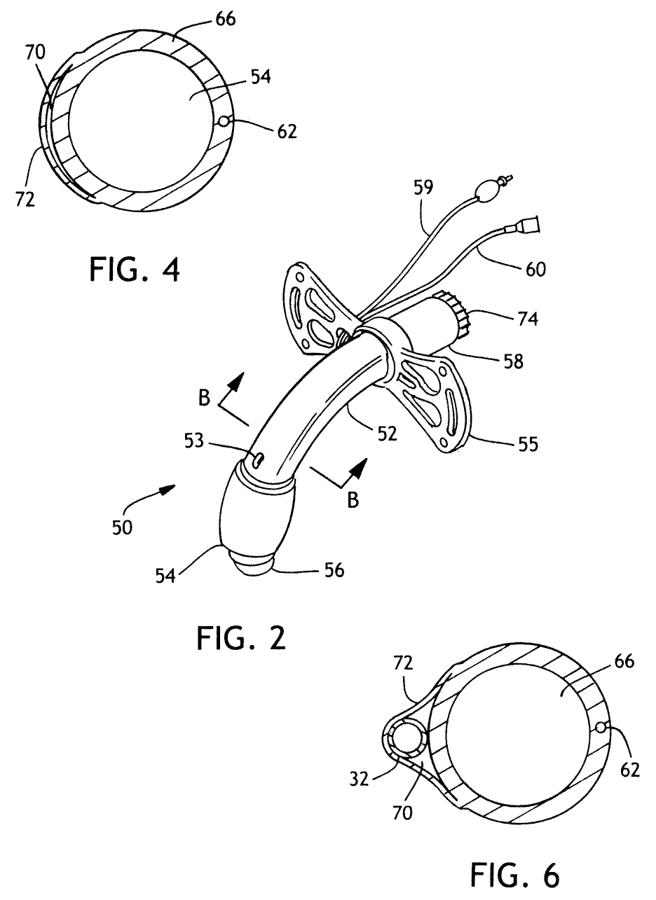

FIG. 2 is an illustration of a cuffed tracheal device 50 having a tube 52, an inflatable cuff 54 and a flange 55 that rests against the outside of the patient's throat when the tracheal device 50 is in place. The tube 52 has a distal end 56 and a proximal end 58. The cuff 54 is inflated by air supplied to an inflation line 60. The tube 52 may connect to a ventilator (not shown) at its proximal end 58 so that breathing air may be delivered to a patient. The tube 52 has a flexible lumen 70 (not visible in FIG. 1) that terminates at a port 53 above the cuff 44 and is accessible through a flexible lumen access tube 59 that begins near the proximal end 58 of the tube 52. FIG. 4 is a cross sectional view of the tube 52 taken at point B-B showing the various lumens within the tube 52. This view illustrates the inflation lumen 62 that is connected on one end to the inflation line 60 and on the other end to the cuff 54 and that delivers the air from the inflation line 60 to the cuff 54. FIG. 4 also shows the ventilating lumen 64. The ventilating lumen 64 is enclosed by the ventilating lumen wall 66. A flexible, collapsible lumen 70 is formed adjacent to and on the outside of the ventilating lumen wall 66 by the flexible wall 72. The flexible lumen 70 terminates and opens onto the outside of the tube 52, just above the cuff 54, where it forms an aperture or port 53. The trach tube 50 may also have a disposable cannula whose proximal end 74 is visible in FIG. 2 and that is placed in the tube 52 to reduce the buildup of biological material. The disposable cannula (not visible) runs inside the tube 52 and fits within the ventilating lumen 64.

When an endotracheal tube or trach tube is placed in the trachea of a patient, secretions that are produced by the body travel down the walls of the trachea and eventually accumulate on top of the cuff. These secretions are a breeding ground for bacteria. Should these secretions pass by the cuff and travel into the lungs, they may cause the development of ventilator acquired pneumonia in the patient. It is important, therefore, to remove the secretions so that complications may be avoided.

One way of removing secretions is described in the Summary and is to insert a suction catheter into the flexible lumen described herein. The flexible lumen terminates just above the cuff where it opens on the outside of the ventilating lumen at a port. The suction catheter is connected to a vacuum source on the end of the suction catheter outside the patient. The suction catheter is inserted into the flexible lumen at the flexible lumen's proximal end and the flexible wall readily bends or moves, which easily allows the suction catheter to pass downwardly through the flexible lumen to the port so that the suction catheter may suck the secretions out of the space above the cuff. Once the secretions have been removed the suction catheter may be withdrawn. This allows the flexible wall to move back into position and lie against the ventilating wall, thus enlarging the ventilating lumen to provide the patient maximum breathing air flow with minimum resistance.

It is important to minimize the intrusions into the ventilating lumen cross sectional area so that resistance to air flow is not increased. The devices described herein accomplish that goal. By way of comparison, for example, a commercially available endotracheal tube having a dedicated suction or “evac” lumen from Mallinckrodt Inc. has an inner diameter (ID) of 8.0 millimeters and an outer diameter (OD) of 11.8 mm. An endotracheal tube made according to the description in the Summary may have the same ID but may be at least 5 percent smaller in OD, more particularly at least 7.5 percent smaller in OD and still more particularly at least 9 percent smaller in OD.

Furthermore, a tracheal tube using the cuffs taught in U.S. Pat. Nos. 6,526,977 or 6,802,317 results in much less leakage past the cuff into the lungs than conventional thick cuffs allow. The '977 and '317 cuffs are desirably made from a soft, pliable polymer such as polyurethane, polyethylene teraphihalate (PETP), low-density polyethylene (LDPE), polyvinyl chloride (PVC), polyurethane (PU) or polyolefin. The cuff should be very thin; with a thickness on the order of 25 microns or less, e.g. 20 microns, 15 microns, 10 microns or even as low as 5 microns in thickness, though at least 1 micron. The cuff should also desirably be a low pressure cuff operating at an inflation pressure of about 30 mmH₂O or less, such as 25 mmH₂O, 20 mmH₂O, 15 mmH₂O or less. Suitable cuffs are described in U.S. Pat. Nos. 6,802,317 and 6,526,977.

U.S. Pat. No. 6,802,317 describes a cuff for obdurating a patient's trachea as hermetically as possible, comprising: a cuffed balloon which blocks the trachea below a patient's glottis, an air tube, the cuffed balloon being attached to the air tube and being sized to be larger than a tracheal diameter when in a fully inflated state and being made of a soft, flexible foil material that forms at least one draped fold in the cuffed balloon when inflated in the patient's trachea, wherein the foil has a wall thickness below or equal to 0.01 mm and the at least one draped fold has a loop found at a dead end of the at least one draped fold, that loop having a small diameter which inhibits a free flow of secretions through the loop of the at least one draped fold.

U.S. Pat. No. 6,526,977 teaches a dilator for obdurating a patient's trachea as hermetically as possible, comprising a cuffed balloon which blocks the trachea below a patient's glottis, an air tube, the cuffed balloon being attached to the air tube and being sized to be larger than a tracheal diameter when in a fully inflated state and being made of a sufficiently soft, flexible foil material that forms at least one draped fold in the cuffed balloon when fully inflated in the patient's trachea, wherein the at least one draped fold formed has a capillary size which arrests free flow of secretions across the balloon by virtue of capillary forces formed within the fold to prevent aspiration of the secretions and subsequent infections related to secretion aspiration.

Since the '977 and 317 cuffs inhibit or arrest the free flow of secretion past the cuff, the secretions build up above the cuff and discontinuous or intermittent suctioning may be used. Intermittent suctioning is much safer for the tracheal wall since it reduces the chance that the suction lumen inlet will adhere to the wall and subject it to the force of suction. In addition, the possibility that the suction lumen will clog between suctionings is far less for tubes with flexible wall lumens than for tubes with dedicated suction lumens since the flexible lumen will close when the suction catheter is removed and the secretions will not be able to remain in the lumen. This ensures that a clear suction lumen will be available each time it is needed.

FIGS. 5 and 6 illustrate the behavior of the flexible wall when a suction catheter is inserted. FIG. 5 is a cross sectional view of the endotracheal device 10 of FIG. 1 taken at point A-A after the insertion of a suction catheter 32 into the flexible lumen 30. It can be seen that the flexible lumen wall 28 bends toward the center of the ventilating lumen 24 in order to allow the catheter 32 to enter the flexible lumen 30 when the suction catheter 32 is inserted into the flexible lumen 30 via the flexible lumen access tube 19. In like manner, FIG. 6 shows a cross section of the tracheal device 50 of FIG. 4 taken at point B-B after the insertion of a suction catheter 32 into the flexible lumen 70. It can be seen that the flexible lumen wall 72 bends away from the ventilating lumen 66 in order to allow the catheter 32 to enter the flexible lumen 70 when the suction catheter 32 is inserted into the flexible lumen 70 via the flexible lumen access tube 59.

The flexible lumen wall should be smaller in thickness than the ventilating lumen wall and should be less than a millimeter in thickness, more particularly less than 0.5 mm in thickness, to allow it to bend. The size or cross-sectional area of the flexible lumen will be dependent upon the size of the suction catheter that is to be inserted into it. As seen in FIGS. 3 and 4, however, the flexible lumen area may assume a crescent shape when a suction catheter is not inserted. This crescent shape may cover between a quarter and as much as one half of the inner or outer circumference of the ventilating lumen, more particularly about a third of the circumference of the ventilating lumen. The flexible lumen may be coated internally with a lubricious substance to reduce the friction during the insertion of the suction catheter. Alternatively or in addition, the suction catheter may be coated with such a lubricious substance. A suitable lubricious substance is for example, a polyethylene glycol (PEG) or other such bio-acceptable material known to those skilled in the art.

Suction catheters are well known and widely commercially available for many medical uses. Suction catheters are long, flexible tubes used to remove secretions from the airway and are available in many sizes, commonly from 10 to 20 French and varying lengths, typically from 15 to 25 inches (38 to 64 cm). Suction catheters may be made from latex and other polymers.

Suctioning may be performed using an “open” or “closed” system. In the open system, the suction catheter is merely a flexible plastic tube that is inserted into the flexible lumen with a source of suction connected to the proximal end of the suction catheter. Anything that the suction catheter touches before entering the lumen must be maintained in a sterile condition so a “sterile field” must be created on or next to the patient. The suction catheter must be carefully handled after it is used since it will be coated with the patient's secretions. In contrast, in the “closed” system, for example that disclosed in commonly owned U.S. Pat. No. 4,569,344, a device which may be used to suction secretions is enclosed within a generally cylindrical plastic bag to eliminate or minimize contamination of the suction catheter prior to use. This is generally referred to as a “closed suction catheter” and is available under the tradename TRACH CARE® from BALLARD® Medical Products (Kimberly-Clark Corporation). As the patient requires artificial removal of secretions, the suction catheter may be advanced through one end of the plastic bag, through a connecting fitting and into the flexible lumen. The other, proximal end of the suction catheter is attached to a source of suction. Suction may be applied using, for example, a finger controlled valve on the proximal end of the suction catheter, and the secretions removed. Secretions are thus drawn into the lumen of the suction catheter tube and removed and the system remains closed. The suction catheter is subsequently withdrawn from the flexile lumen and back into the plastic bag to keep the circuit closed. Closed suction systems are generally preferred by healthcare providers since the provider is better protected from the patient's secretions. Closed suction systems are also easier and quicker to use since a sterile field need not be created each time the patient must be suctioned, as is required in open suction systems.

The closed suction catheter may be permanently attached to the proximal end of the flexible lumen or may be detachably connected so that it may be replaced periodically. The closed suction catheter, for example, may be releasable connected to the flexible lumen access tube by means of bayonet or luer-type fittings. In this manner, the suction catheter may be removed and disposed of periodically to assist in removing sources of infection from the patient. A suitable releasable connection is a novel quick connect fitting having male and female fitting ends and a tapered internal luer-type seal as described in co-assigned, co-pending patent application Ser. No. ______, attorney docket number 64496506, filed on the same day as the instant application and incorporated by reference. The male fitting end has a periphery upon which is mounted at least one boss. There may desirably be two bosses on the periphery of opposite sides of the male fitting end, and they may be of different lengths. The female fitting end has a slot into which the boss may be inserted. At the bottom of the slot is a stop to limit the insertion depth of the boss.

The male and female ends may then be rotated relative to each other to move the boss into a window on the female end. The window has a frame and the upper frame is angled slightly which serves to draw the male end farther into the female end. The window has a side frame that stops the rotational movement of the boss. When the movement of the boss is stopped, the male and female tapers are in substantially leak-free contact. The boss on the male fitting end may desirably be at an a downward angle between 5 and 15 degrees, more particularly between 7 and 12 degrees and still more particularly between 9 and 10 degrees, relative to the perpendicular of the centerline of the fitting. The male and female fitting ends may be rotated relative to each other in a right hand turn orientation to tighten them, desirably for about a quarter turn though more or less may be desirable in particular applications. A left hand turn orientation may also be used if desired. In usage, once the boss of the male fitting end is inserted into the slot of the female fitting, it may advance only so far as to contact the stop at the bottom of the slot. The stop is placed at the proper depth so as to bring the luer tapers of the male and female fittings close together or into contact. Once the boss is fully inserted into the slot, the male fitting end may be rotated in only one direction relative to the female fitting to move the boss into position in the window. As the boss moves into the window, contact with the upper (angled) frame of the window causes the entire male fitting end to move slightly farther into the female fitting end. When the boss contacts the far window side frame, movement is stopped and the tapers of the male fitting end and the female fitting end are fully engaged and are in substantially leak-free contact.

The tracheal catheter described herein may be made from polymeric materials by conventional extrusion or injection molding techniques known to those skilled in the art. The tracheal catheter may be extruded through a die having a pattern like that shown in the cross sectional views in the Figures. As the tracheal catheter exits the die, it cools and the shape becomes more permanent. The just-produced tracheal catheter may be subjected to differing pressures within the various lumens and on the outside of the tracheal catheter in order to help stabilize the tube as it cools. Such pressure variations are within the normal range of experimentation for such manufacturing processes and may be readily developed by one skilled in the art.

Polymers suitable for the production of the tracheal catheter include polyvinyl chloride, polyurethane and polyolefins like polyethylene and polypropylene. Nylons and, polyethylene terephthalate (PET) materials may also be used through their cost may be prohibitive. Blends of suitable polymers may also be used. It is also possible using known extrusion techniques to extrude parts of the tracheal catheter from one polymer and other parts of the tracheal catheter from other polymer. For example, the ventilating lumen walls may be made of a first polymer like polyvinyl chloride and the flexible wall may be made from a second polymer like polyurethane. One particularly suitable polymer is a polyvinyl chloride commercially available from Colorite Polymers Inc. as 8566G-015.

Once the tracheal catheter is formed and cooled, it may be cut to the appropriate length and a balloon cuff attached to it by conventional means. An opening port or aperture must be made into the flexible lumen on the outside surface of the tracheal catheter (an exterior surface) on the side away from the tracheal catheter and proximal to (above) the cuff for future suctioning. The proximal end of the flexible lumen terminates at or near the proximal end of the tracheal catheter and is adapted to allow the insertion of a catheter, e.g. a suction catheter. A removable closure cap may be provided for the flexible lumen to cover and close it while it is not in use to avoid contamination. The cap may be tethered to the tracheal catheter to so that it is not misplaced. The inflation lumen must be connected to a length of tubing in the conventional manner so that it may be attached to a source of inflating air.

Modifications and variations of the presently disclosed device and method will be obvious to those of skill in the art from the foregoing detailed description. For example, thought the discussion above mentions the insertion of suction catheter into the flexible lumen, other catheters and devices, such as cameras or other viewing devices may be inserted into the flexible lumen as well provide they are of the appropriate size. Such modifications and variations are intended to come within the scope of the following claims.

1. A tracheal catheter tube comprising:
a tube having ventilating lumen and having a proximal end and a distal end portion, wherein the distal end portion is adapted for insertion into a trachea;
an inflatable balloon surrounding the tube near the distal end of the tube, which balloon upon inflation blocks a remainder of the trachea;
means for inflating and deflating the balloon;
a flexible wall adjacent said ventilating lumen, said flexible wall defining a flexible lumen, said flexible lumen adapted to bend to allow insertion of a catheter into said flexible lumen;
said flexible lumen terminating near and proximal to said balloon and having an aperture on an outside surface of said tube;
wherein said flexible wall lies substantially flat against a wall of said ventilating lumen when said catheter is not present.

2. The tracheal catheter of claim 1, wherein said flexible lumen has a proximal termination near the proximal end of the tube and the catheter may be inserted into the flexible lumen at the proximal termination of said flexible lumen.

3. The tracheal catheter of claim 2 wherein said balloon is sized to be larger than a tracheal diameter when in a fully inflated state and being made of a sufficiently soft, flexible foil material that forms at least one draped fold in the cuffed balloon when fully inflated in the patient's trachea, wherein the at least one draped fold formed has a capillary size which inhibits a free flow of secretions across the balloon by virtue of capillary forces formed within the fold

4. The tracheal catheter of claim 3 wherein the foil has a wall thickness below or equal to 25 microns.

5. The tracheal catheter of claim 1, wherein the tube is made in part from a polymer selected from the group consisting of polyvinyl chloride, polyurethane, polyethylene, polypropylene, nylon, polyethylene terephthalate and blends and mixtures thereof.

6. An endotracheal tube comprising:
a hollow ventilating tube having a proximal end and a distal end, wherein the distal end portion is adapted for insertion into the tracheal lumen through the oral cavity;
an inflatable sealing cuff surrounding the hollow tube towards the distal end of said tube, which cuff upon inflation expands to occlude the tracheal lumen outside the tube;
means for inflating and deflating the cuff;
a flexible wall within said hollow tube, said flexible wall defining a flexible lumen and adapted to bend to allow insertion of a suction catheter into said flexible lumen from the proximal end of said flexible lumen;
said flexible lumen terminating proximal to said balloon and having an aperture on an exterior surface of said hollow tube;
wherein said flexible wall lies substantially flat against an interior wall of said hollow tube when said suction catheter is not present.

7. The endotracheal tube of claim 6 wherein said suction catheter is enclosed within a plastic bag when not inserted into the flexible lumen.

8. The endotracheal tube of claim 6 wherein the tube is made in part from a polymer selected from the group consisting of polyvinyl chloride, polyurethane, polyethylene, polypropylene, nylon, polyethylene terephthalate and blends and mixtures thereof.

9. The endotracheal tube of claim 6 wherein said flexible wall has a thickness less than 1 mm.

10. The endotracheal tube of claim 6 further comprising a lubricious substance within said flexible lumen or on said suction catheter.

11. A method of providing suction to the subglottic space of an intubated patient comprising the steps of;
providing a hollow tube having a proximal end and a distal end, wherein the distal end portion is arranged for insertion into the tracheal lumen, said tube having an inflatable sealing cuff with a thickness of 25 microns or less surrounding the hollow tube towards the distal end of said tube, which cuff upon inflation expands to occlude the tracheal lumen outside the tube, said tube further having a flexible wall adjacent said hollow tube, said flexible wall defining a flexible lumen and adapted to bend to allow insertion of a catheter into said lumen from the proximal end, said flexible lumen terminating proximal to said balloon and having an aperture on an exterior surface of said hollow tube so that secretions may be suctioned through said aperture, said flexible wall lying substantially flat against a wall of said hollow tube when said catheter is not present;
intubating a patient with said tube;
inserting a catheter into said flexible lumen from the proximal end;
applying suction to said catheter.

12. The method of claim 10 wherein said catheter is inserted into said lumen intermittently.
